(19) United States
(12) Patent Application Publication
(10) Pub. No.: US 2022/0184823 A1
(43) Pub. Date: Jun. 16, 2022

(54) PROTECTIVE DRAPE FOR ROBOTIC SYSTEMS

(71) Applicant: THINK SURGICAL, INC., Fremont, CA (US)

(72) Inventors: Daniel P. Bonny, Fremont, CA (US); Joel Zuhars, Fremont, CA (US); Saleh Tabandeh, Fremont, CA (US); Timothy Pack, Fremont, CA (US); Randall Hanson, Fremont, CA (US); Michael E. Hoppe, Fremont, CA (US); Nathan A. Netravali, Fremont, CA (US); Avery N. Goldstein, Fremont, CA (US)

(73) Assignee: THINK SURGICAL, INC., Fremont, CA (US)

(21) Appl. No.: 17/686,617

(22) Filed: Mar. 4, 2022

Related U.S. Application Data
(63) Continuation-in-part of application No. 15/744,253, filed on Jan. 12, 2018.
(60) Provisional application No. 62/196,073, filed on Jul. 23, 2015.

(51) Int. Cl. B25J 19/00; A61B 46/10; A61B 34/30

(52) U.S. Cl. CPC B25J 19/0075; A61B 34/30; A61B 46/10

(57) ABSTRACT

A protective drape for a robotic arm is provided. The protective drape may be used with robotic arm that are required to operate in varied environments that illustratively include industrial applications, a sterile surgical suite for patient care, and a clean room for manufacturing sensitive electronic components. In each of these applications, there is a need to prevent contaminants from infiltrating from the environment to the robot and affecting operation of the robot itself or the robotic system, as well to prevent contaminants from the robot from infecting a patient or contaminating an assembly or process product.

### CROSS REFERENCE TO RELATED APPLICATIONS

This application is a continuation in part application of U.S. application Ser. No. 15/744,253 filed 12 Jan. 2018 that in turn is a U.S. National phase application of International Application Ser. No. PCT/US2016/4278 6filed 18 Jul. 2016 that claims priority benefit of U.S. Provisional Application Serial Number 62/196,073 filed 23 Jul. 2015; the contents of which are hereby incorporated by reference.

### FIELD OF THE INVENTION

The present invention generally relates to robotic systems, and more specifically to a protective drape for robotic systems.

### BACKGROUND OF THE INVENTION

Robotic systems have been developed to aid in a variety of different applications ranging from the industrial, medical, and military fields. Robotic systems have unique characteristics to perform different tasks depending on the application to be performed, where the size, weight, geometry, construction, controls, programs and functionality are all characteristics considered when designing a robot.

Robotic systems are required to operate in varied environments that range from industrial applications to a sterile surgical suite for patient care or a clean room for manufacturing sensitive electronic components. In each of these applications there is a need to prevent contaminants from infiltrating the robot and affecting operation, as well to prevent contaminants from the robot infecting a patient or contaminating product being assembled or tested by the robot. Furthermore, in surgical or medical environments sterile conditions are required to be maintained to prevent the transfer of infectious agents between successive patients being treated by a robot.

A robotic system used in surgery must either be sterile or covered by a sterile drape, while an industrial robot often has a similar protective cover. Often, part of the robotic system must extend beyond the drape so that those parts can interact with the environment beyond the drape to perform desired tasks. In a robotic system with a large range of motion, fixing a drape to the operational end of the robotic system often leads to drape constriction. The resulting twisting and tightening of the drape around the robotic system requires manual adjustments of the drape. This may be alleviated by using a protective drape that is larger than needed to cover the system. With a larger protective drape the robot manipulator has more room to articulate reducing the chances of drape constriction. However, this excess material can interfere with the surgical procedure and use of the device. In addition, a larger protective drape requires additional drape material that is counterintuitive to install since the drape may not resemble the shape of the robot. On the other hand, a protective drape that has a conforming shape to the robot can be difficult to maneuver around the robot during installation.

Currently, tubes that are used to deliver fluid, such as sterile irrigation fluid, to the end effector of the robot must be routed around a protective drape after the drape is installed. This can be a difficult process, particularly keeping the irrigation tube sterile during installation and having the tube oriented in the correct direction. Additionally, exchange of such tubes is often required between surgical procedures owing to the external placement.

Thus there is a need in the art for protective draping for robotic systems that are easy to install, does not restrict the robots movement, and allows the robotic system to perform a specified task.

### SUMMARY OF THE INVENTION

A protective drape for a robotic system is provided that includes a protective tube encapsulating at least a portion of the robotic system with a first ring connected to, or in proximity to a first end perimeter of the protective tube and adapted to fit around a distal portion of the robotic system. A second ring is connected to a second end perimeter of the protective tube, where the second ring is configured to fit around a proximal portion of the robotic system without constraining the rotation of a robotic arm. One or more draw cords are provided to remove excess slack at different points along the length of the protective tube and the robotic arm.

A protective drape for a robotic system is provided that includes a plurality of drape segments adapted to collectively encapsulate at least a portion of the robotic system, with one or more sectional rings positioned between and joining the plurality of drape segments. A first ring is connected to a first end perimeter of the collective drape segments, where the first ring is configured to fit around a distal portion of the robotic system, and a second ring is connected to a second end perimeter of the collective drape segments, where the second ring is configured to fit around a proximal portion of the robotic system.

A protective drape for a robotic system is provided that includes a protective tube having a length configured to encapsulate a robotic arm. A first ring is connected to a first end perimeter of the protective tube, where the first ring is configured to fit around a distal portion of the robotic arm, and a second ring is connected to a second end perimeter of the protective tube, where the second ring is configured to fit around a proximal portion of the robotic arm, and an irrigation, vacuum, or air tube/line is integrated with the protective tube.

A method is provided for deploying a protective drape to encapsulate a robotic system, where the method includes holding a tube of the protective drape alone or connected to at least one of a pair of rings in front of the robotic system; and automatically moving or advancing a robotic arm of the robotic system through the protective tube to encapsulate the robotic system.

### BRIEF DESCRIPTION OF THE DRAWINGS

The present invention is further detailed with respect to the following drawings that are intended to show certain aspects of the present invention, but should not be construed as a limit on the practice of the present invention.

FIG. 1 is a perspective view of a robotic system with a transparent protective drape joined to a robotic arm with rotationally unconstrained rings at the fixed base of the robot and at the rotating tool attachment of an end effector in accordance with embodiments of the invention;

FIG. 2 is a perspective view of a robotic system with a transparent protective drape joined to a robotic arm that includes a blower to control a desired pressure within the protective drape in accordance with embodiments of the invention;

FIG. 3 is a perspective view of a protective drape in a folded state to aid in transportation and to facilitate installation in accordance with embodiments of the invention;

FIG. 4 is a perspective view of a robotic system with a segmented transparent protective drape joined with sectional rings, and joined to a robotic arm with rotationally unconstrained rings at the fixed base of the robot and at the rotating tool attachment point of an end effector in accordance with embodiments of the invention;

FIG. 5 is a perspective view of a robotic system with a segmented transparent protective drape joined with sectional rings, where the sectional rings are joined to the robot arm, and the ends of the protective drape are joined to a robotic arm with rotationally unconstrained rings at the fixed base of the robot and at the rotating tool attachment point of an end effector in accordance with embodiments of the invention;

FIG. 6 is a perspective view of a robotic system with a transparent protective drape with draw cords to remove excess slack at different points along the length of the protective drape, the protective drape joined to a robotic arm with rotationally unconstrained rings at the fixed base of the robot and at the rotating tool attachment point of an end effector in accordance with embodiments of the invention;

FIG. 7 is a perspective view of a robotic system with a transparent protective drape with an irrigation, vacuum, or air tube/line forming a skeleton along the length of the protective drape, the protective drape joined to a robotic arm with rotationally unconstrained rings at the fixed base of the robot and at the rotating tool attachment point of an end effector in accordance with embodiments of the invention; and

FIG. 8 is an illustrative depiction of a protective drape with two protective tubes to control fluid flow for temperature control in accordance with embodiments of the invention.

FIGS. 9A-9C depict a protective drape in accordance with embodiments of the invention, where FIG. 9A is an assembled view of the protective drape, FIG. 9B is an exploded view thereof, and FIG. 9C is a cross-sectional view of a rotationally unconstrained ring and a portion of a protective tube.

FIGS. 10A-10C depict a protective drape in accordance with embodiments of the invention, where FIG. 10A is an assembled view of the protective drape, FIG. 10B is an exploded view thereof, and FIG. 10C is a plan view of a rotationally unconstrained ring.

### DETAILED DESCRIPTION OF THE INVENTION

The present invention has utility as a protective draping for a robot of a robotic system. The following description of various embodiments of the invention is not intended to limit the invention to these specific embodiments, but rather to enable any person skilled in the art to make and use this invention through exemplary aspects thereof.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

Embodiments of the present invention describe a protective drape for, and method of usage with a robotic system. It should be appreciated that any autonomous (i.e., active), semi-autonomous, passive, or haptic robotic system either for medical or industrial applications may benefit from the system and methods disclosed herein. Embodiments of the invention may be used with robotic systems that are required to operate in varied environments that illustratively include industrial applications, a sterile surgical suite for patient care, and a clean room for manufacturing sensitive electronic components. In each of these applications where embodiments of the invention are employed, there is a need to prevent contaminants from infiltrating from the environment to the robot and affecting operation of the robot itself or the robotic system, as well to prevent contaminants from the robot from infecting a patient or contaminating an assembly or process product. Furthermore, in surgical or medical environments sterile conditions are required to be maintained to prevent the transfer of infectious agents between patients being treated by the robots; the present invention addresses these requirements with the deployment of a protective drape that is different and superior to the prior art.

Embodiments described herein make reference to a robotic system. It should be understood that the robotic system may further include external components such as external hardware and software, tracking systems, external user interfaces and external user input mechanisms. The external components may require additional protective draping independent of the robotic system described herein, unless otherwise stated. Examples of the components and control of a robotic system are described in U.S. patent application Ser. No. 12/703,125 and U.S. Pat. No. 5,806,518. Examples of such robotic systems include the LBR iiwa Lightweight Industrial Robot Series (KUKA Robotics Corp., Shelby Township, Mich.), the ROBODOC® Surgical System (THINK Surgical, Inc., Fremont, Calif.), and the RIO® Robotic System (Mako Surgical Corp., Ft. Lauderdale, Fla.).

Embodiments of the protective drape may be made of paper, woven materials, fabrics, plastic films, plastic sheets, foils, and combinations thereof. In certain inventive embodiments, an inventive drape is formed with transparent plastic film to allow visual inspection of an encapsulated robot without compromising the position or integrity of the protective drape.

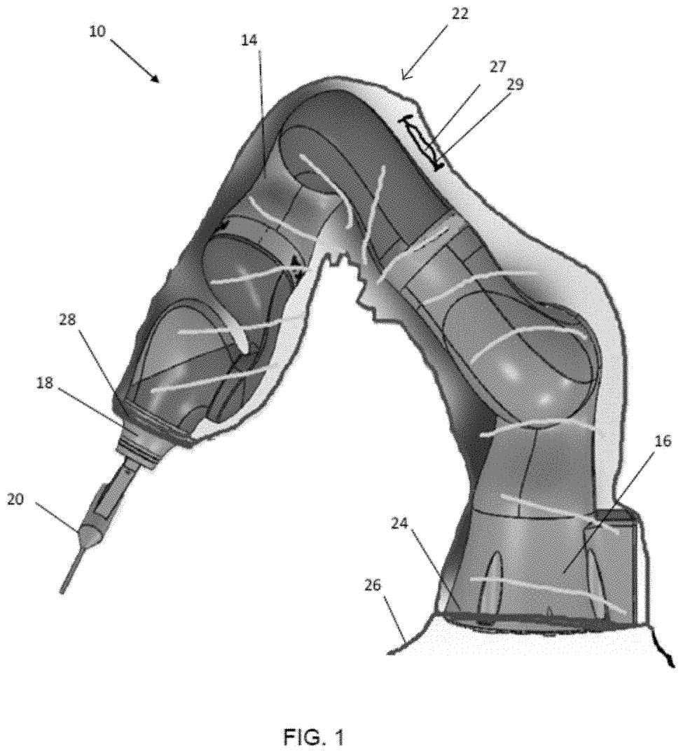

Referring now to the figures, FIG. 1 depicts a draped robotic system 10 in accordance with an embodiment of the invention. The robotic system 10 may include a segmented robotic arm 14 that rotates on a base 16, and an end effector manipulator 18 (i.e., a most distal segment/link of the robotic arm 14) that rotates relative to the robotic arm 14. A most distal portion of the robotic system 10 is an end effector 20 that may be removably attached to the end effector manipulator 18. An end-effector 20 may refer to a tool that directly interacts with the environment, and may also refer to one or more components associated with a tool. Examples of an end-effector include a cutter, an end-mill, a drill bit, and a saw, as well as one or more components associated with the tool such as a housing/casing for a tool, a handle of a tool, a motor for driving a tool, or other components associated with a tool. An end-effector 20 may also refer to a device, an apparatus, or an assembly of components that attaches to a most distal segment/link of a robotic arm 14. The most proximal portion of the robotic system 10 is the base 16. The segments (or links) of the robotic arm 14 may be connected by various revolute, prismatic or spherical joints to actuate the end effector 20 in one or more degrees of freedom, preferably five or more. The protective drape 22 is formed of a sheet material formed as a tube 25 that is joined to the robotic system 10 with rotationally unconstrained rings 24 and 28. In a specific inventive embodiment, the ring 28 may be joined to a distal portion of the robotic system 10, such as the end effector 20, or the end effector manipulator 18. Ring 24 may be joined to a more proximal portion of the draped robotic system 10 such as the base 16 or a proximal segment of the robotic arm 14. For the purposes of visual clarity, the drape 22 is depicted as being formed of a transparent material, such as polyethylene; however, it is appreciated that paper, woven materials, fabrics, non-transparent plastic films or sheets, foils, and combinations thereof are also suitable for the formation of the drape 22. The protective tube 25 that forms the protective drape 22 may be formed according to the shape and size of the robotic system 10. For example, the protective tube 25 may be formed or manufactured in the shape of a hollow cylinder, hollow square, or of any hollow irregular shape depending on the type or geometry of the robotic system 10. Additionally, the protective tube 25 may be readily formed by joining opposing sheet edges to define, for example a hollow cylinder, or may be formed by a seamless film or foil, by conventional techniques. It is also appreciated that luminal segments of like or different material are readily joined to form a segmented protective drape. Alternatively, swatches of sheet material may be joined to form a tube 25 with a quilt-like pattern.

The rings (24, 28) may be formed of rigid or semi-rigid materials illustratively including metals or polymers. The rings (24, 28) may or may not be shaped and sized according to the shape and size of the portions of the robotic arm where the rings (24, 28) are adapted to reside. For example, the rings (24, 28) may be circular as depicted throughout the drawings, or confer other geometries including ovals, polygons, or organic shapes. The ends of the protective tube 25 may be joined to the rings (24, 28) using techniques known in the art (e.g., adhesives, fastening elements (clasps, clamps, clips, screws, pins, hook and loop, etc.), shrink wrapped, heat wrapped, stitched, etc.).

In some inventive embodiments, the ring 28 engages the end effector 20. The use of rotationally unconstrained rings (24, 28) allows the drape 22 to be attached to the end effector 20 directly rather than a link proximal to the last revolute joint, while the end effector 20 is free to rotate an unlimited number of rotations without constricting the drape 22, and allows the wrist to perpetually invert while holding the end effector position for an unlimited number of rotations. The use of rings reduces the size of the drape for easy installation.

In some inventive embodiments, the drape 22 has an opening 27 in a preselected position to provide access to the arm 14 or base 16. A seal 29 provides for closure of the opening 27. The seal 29 includes a contact adhesive strip, or a fastening structure as detailed in U.S. Pat. No. 5,809,621. Typically the drape is a continuous sheet with adhesives to combine multiple drape components. However, having an opening with a seal can be advantageous if there are controls on the arm or on the base that require access. It is appreciated that a seal 29 is readily formed such that a sterile barrier exists between the robot 12 and the exterior environment.

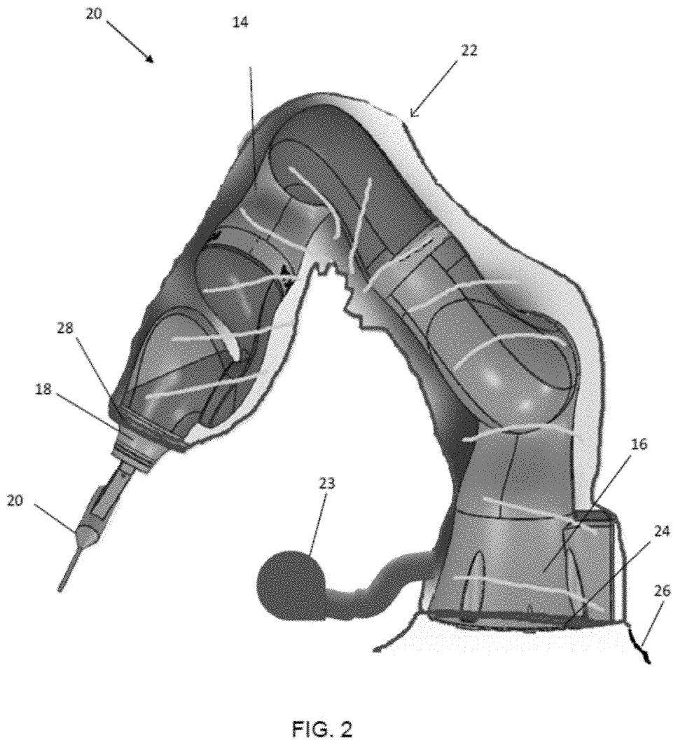

In still other embodiments of the present invention, with respect to FIG. 2, a robotic system 15 includes a blower, vacuum, or air compressor 23 to control the pressure within the protective drape 22. Depending on the nature of the work environment, the draped robotic system 10 has either a positive pressure or negative pressure within the protective drape 22 relative to the surrounding environment. By way of example, in an industrial setting with corrosive or particle ladened environment, a blower 23 may be used to create positive pressure inside the drape 22, protecting the robotic system 15 and further serves to push the sheet material of the drape 22 away from moving components of the robotic system 15. In a medical setting, it may be advantageous to have a vacuum 23 to create negative pressure within the protective drape 22 such that particles or debris on the robotic system 15 cannot escape or contaminate the surgical field. In certain inventive embodiments, the robotic system 15 may include a pressure control system to control the direction of airflow. Therefore, depending on the application or stage of a procedure, the area between the robotic system 15 and the protective drape 22 may be toggled between a positive or negative pressure state. Additionally, one or more of the rings (24, 28) may provide a hermetic seal to help control the pressure within the drape 22. Although, a hermetic seal may not be necessary if the pressure differential is sufficient to maintain the pressure state.

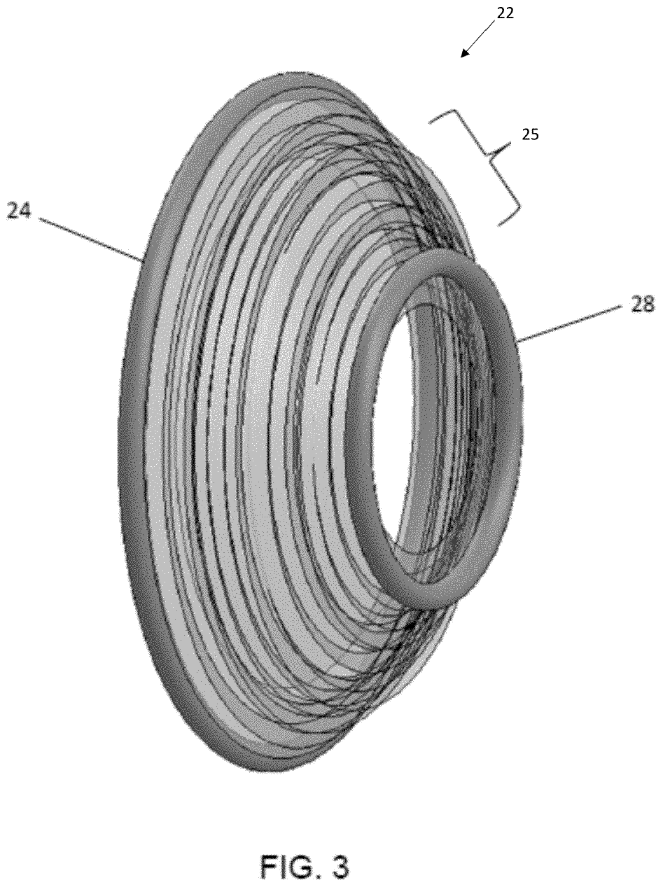

In a particular inventive embodiment, with respect to FIGS. 1 and 3, a set of rings (24, 28), where one ring 28 is approximately the size of the end effector manipulator 18 or end effector 20, or a ring that does not constrain rotation (i.e., a “slip ring”) of the end effector manipulator 18 or end effector 20 is used at the distal end of the protective drape 22. The second ring 24 is a simple ring that is larger than the largest diameter of the robot arm 14 and base 16. The protective drape 22 may be compressed or folded between the two rings (24, 28) as shown in FIG. 3. For example, the protective drape 22 may have an accordion-type fold similar to that of a bellow. When deployed, as shown in FIG. 1, the drape 22 may be shaped like the robotic arm 14 in a manner that the robotic system 10 is able to be moved throughout its entire range of motion without cinching the drape 22. It is appreciated that cinching of the sheet material of the drape 22 can limit the operational range of the robotic system 10 in general, and the end effector 20 in particular; additionally, the barrier function of the drape 22 is compromised if the cinching results in a tear.

In a particular inventive method, the protective drape 22 is installed with a user holding the rings (24, 28) in front of the robotic system 10. The first ring 28 of the drape 22 is fixed to the distal end of the robotic system 10 (end effector 20 if attached, or end effector manipulator 18 if the end effector 20 is not attached) by a user. The robotic arm 14 then automatically moves or advances through the rings (24, 28) in a manner that the user does not need to move the second ring 24. As the robotic arm 14 moves through the second ring 24, the drape 22 unfolds until the user is holding the second ring 24 near the base 16 of the robotic system 10. In a specific embodiment, the user manually unfolds the drape 22 by holding the first ring 28 and moving the second ring 24 down the length of the arm 14. In another embodiment, a separate device (not shown) holds the two rings (24, 28) of the folded drape 22. The robotic system 10 automatically moves the end effector 20 to the device, where the end effector 20 either automatically attaches to the first ring 28, or the user manually attaches the first ring 28 to the end effector. Subsequently, the robotic system 10 either moves the arm 14 through the second ring 24, while the second ring 24 remains stationary, or, the robotic system 10 “picks up” the drape 22, lifts the drape 22 above the arm 14, and allows gravity to unfold the drape 22 along the robotic arm 14. After the second ring 24 is at the base 16 of the robotic system 10, the second ring 24 may be attached to a base drape 26 to create a base seal. The base drape 26 may drape any additional components beyond the base 16 of the robotic system 10, such as a supporting structure supported on the floor or a wall that might contain additional robotic hardware components.

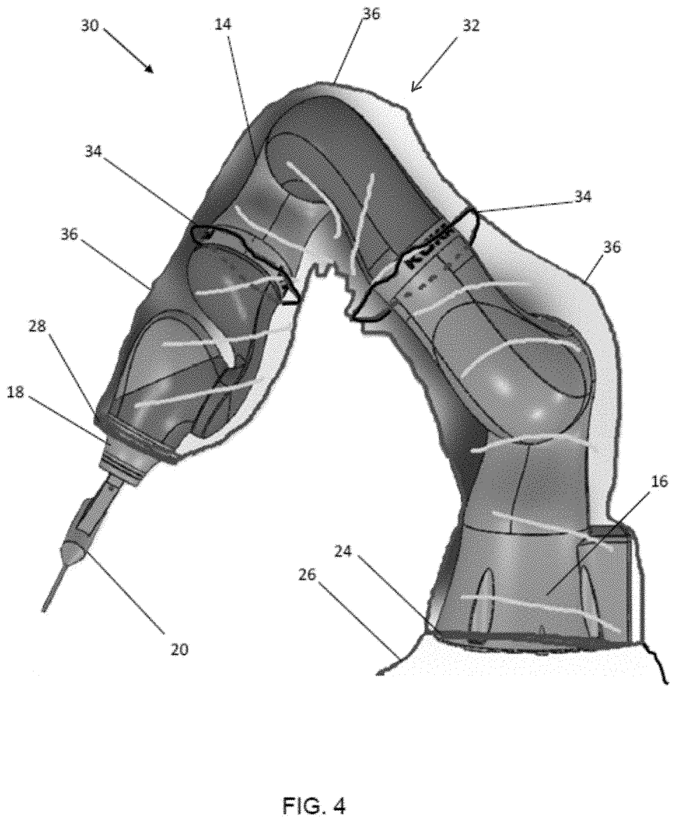

FIG. 4 is a perspective view of a robotic system 30 with a segmented protective drape 32 having drape segments 36 that are joined by sectional rings 34 in accordance with embodiments of the invention. The segmented protective drape 32 is joined to a robotic arm 14 with rotationally unconstrained rings (24, 28) at the fixed base 16 of the robotic system 30, and at the end effector manipulator 18 or end effector 20. With respect to FIG. 4, like numerals have the meaning ascribed thereto with respect to the preceding drawing. In a specific embodiment, the sectional rings 34 may form a rigid connection between the segments 36, or the sectional rings 34 may form a rotationally-unconstrained connection between the segments 36.

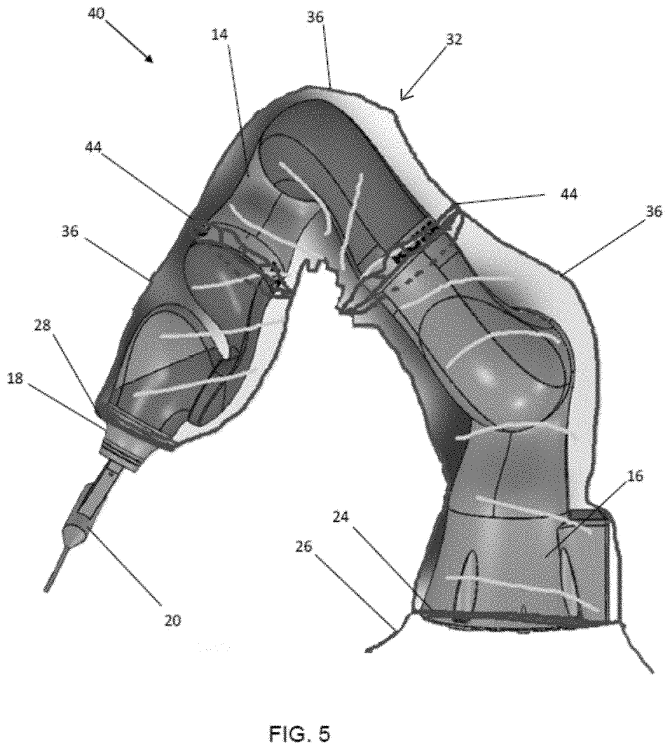

FIG. 5 is a perspective view of a robotic system 40 with a segmented protective drape 32 joined with sectional rings 44, where the sectional rings 44 are joined or clipped onto the robot arm 14 in accordance with embodiments of the invention. The ends of the protective drape 32 are joined to a robotic arm 14 with rotationally unconstrained rings (24, 28) at the fixed base 16 of the robotic system 40, and at the end effector manipulator 18 or end effector 20. With respect to FIG. 5, like numerals have the meaning ascribed thereto with respect to the preceding drawings.

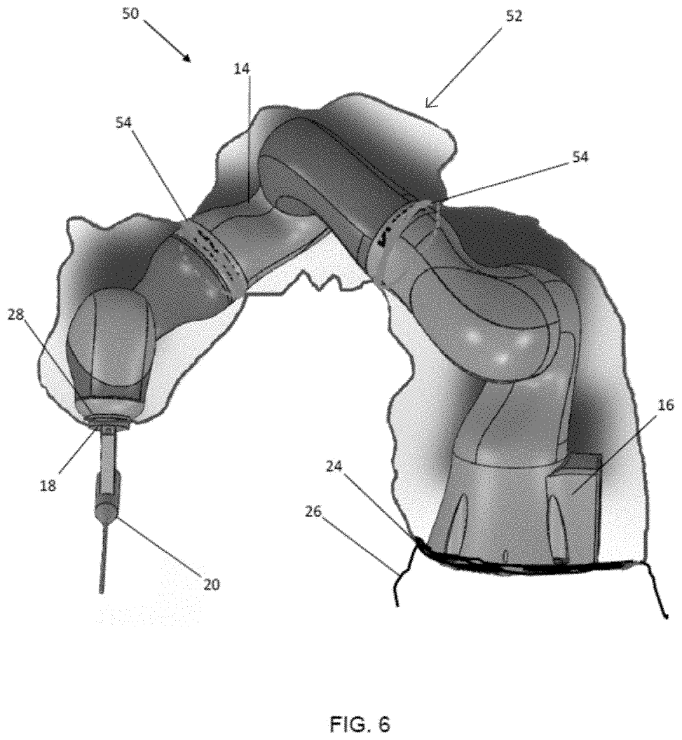

FIG. 6 is a perspective view of an embodiment of a robotic system 50 with a protective drape 52 with draw cords 54 to remove excess slack at different points along the length of the protective drape 52. With respect to FIG. 6, like numerals have the meaning ascribed thereto with respect to the preceding drawings. The protective drape 52 is joined to a robotic arm 14 with rotationally unconstrained rings at the fixed base 16 of the robot 12 and at the end effector manipulator 18 or end effector 20. The use of draw cords 54 allows for a uniform-sized drape with uniform cross-section which is easier to manufacture, while allowing slack where needed but preventing excess slack where it is not needed. In a specific embodiment, a tube-shaped protective drape 52 that has a larger diameter as the largest diameter section of the robotic system 50, and allows the necessary slack to prevent twisting during rotation of the arm 14, has locations along the length of the drape 52 where the cross-section of the robotic arm 14 is smaller and excess slack is not needed, draw cords 54 are placed within the drape 52 to allow the drape 52 to be cinched against the robot 12.

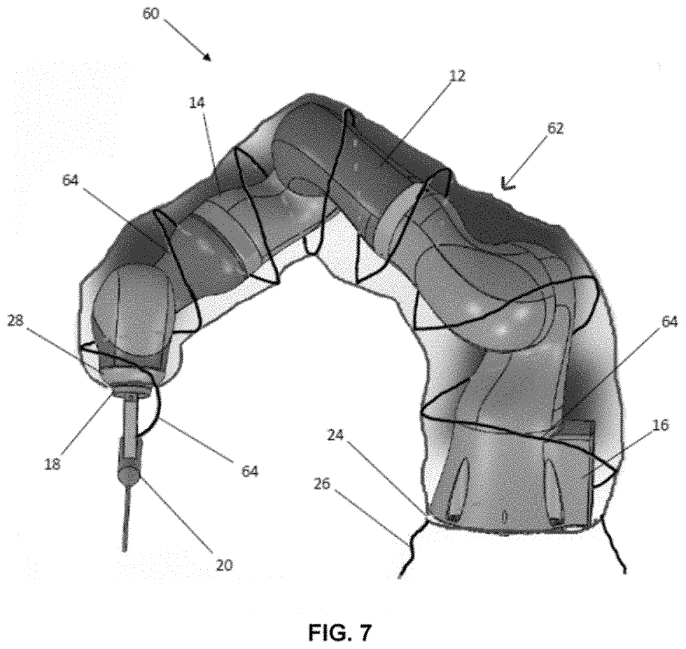

FIG. 7 is a perspective view of a robotic system 60 with a protective drape 62 with an irrigation, vacuum, or air tube/line 64 forming a skeleton along the length of the protective drape 62 in accordance with embodiments of the invention. The protective drape 62 is joined to the robotic arm 12 with rotationally unconstrained rings (24, 28) at the fixed base 16 of the robotic system 60 and at the end effector manipulator 18 or end effector 20. With respect to FIG. 7, like numerals have the meaning ascribed thereto with respect to the preceding drawings. In a specific embodiment the irrigation, vacuum, or air tube/line 64 is in a helical or spring like pattern along the length of the protective drape 62.

In a specific embodiment, the irrigation, vacuum, or air tube/line 64 may come pre-packaged and folded with the drape 62 such that unfolding the drape 62 also unwinds the irrigation tube/line 64 such that the irrigation tube/line 64 is in an optimal position on the robot 12 and ensures that the correct end of the irrigation tube/line 64 is at the end effector 18 when the drape 62 is deployed. In specific embodiments, the irrigation tube 64 may also provide an external “skeleton” to the drape 62, preventing the drape 62 from binding up and facilitating an easier installation of the drape 62. The irrigation tube/line 64 could be coiled such that unfolding the drape 62 keeps the drape 62 coiled around the robot 12.

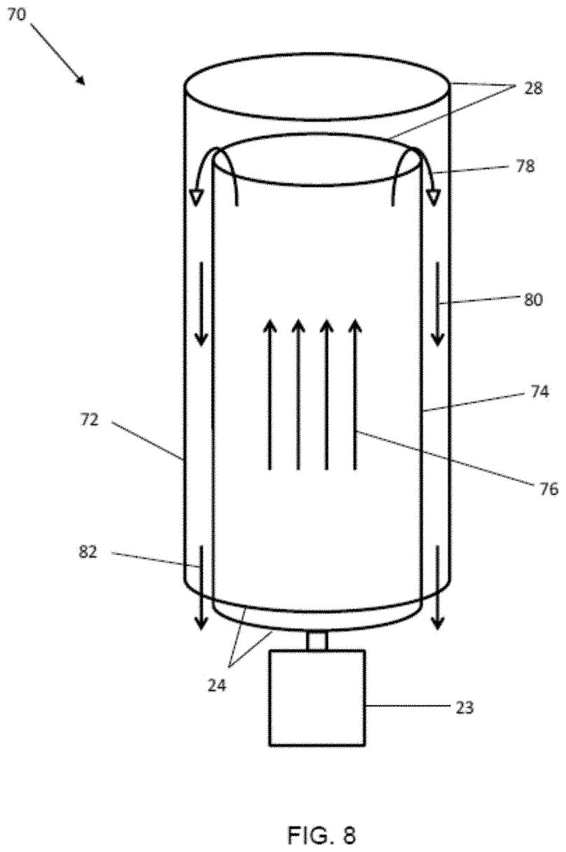

In a particular inventive embodiment, with respect to FIG. 8, a protective drape 70 for a robotic system may include an outer tube 72 and a second inner tube 74 incorporated within, wherein a fluid (e.g., cooling air) can pass there between. A blower 23 may be attached to the inner tube 74 near ring 24 to blow fluid over the components of the robotic system (i.e., direction of arrows 76) encapsulated within the inner tube 74 of the protective drape 70. The fluid may then come out inside of the protective drape 70 near the front of the inner tube 74 and then through the space between the outer tube 72 and inner tube 74 (fluid flow direction is indicated by arrows 78 and 80). The fluid may then exit (indicated by arrow 82) back out near the blower 23 between the inner tube 74 and outer tube 72. The protective drape 70 will keep the temperature of the robotic system within the inner tube 74 down, which otherwise could increase due to the enclosing of the robot hardware.

The rings 24 and 28 of protective drape 70 may be attached to both the inner tube 74 and the outer tube 72. In a specific embodiment, ring 28 is attached to both the inner tube 74 and the outer tube 72, while ring 24 is only attached to the inner tube 74 to allow all the fluid to exit near the blower. If ring 28 is attached to both the inner tube 74 and the outer tube 72, holes or other perforations may exist near the distal portion of the inner tube to allow fluid to pass to the space between the inner tube 74 and outer tube 72. Therefore, the fluid may not expel near the end effector 20 where the surgical procedure is performed.

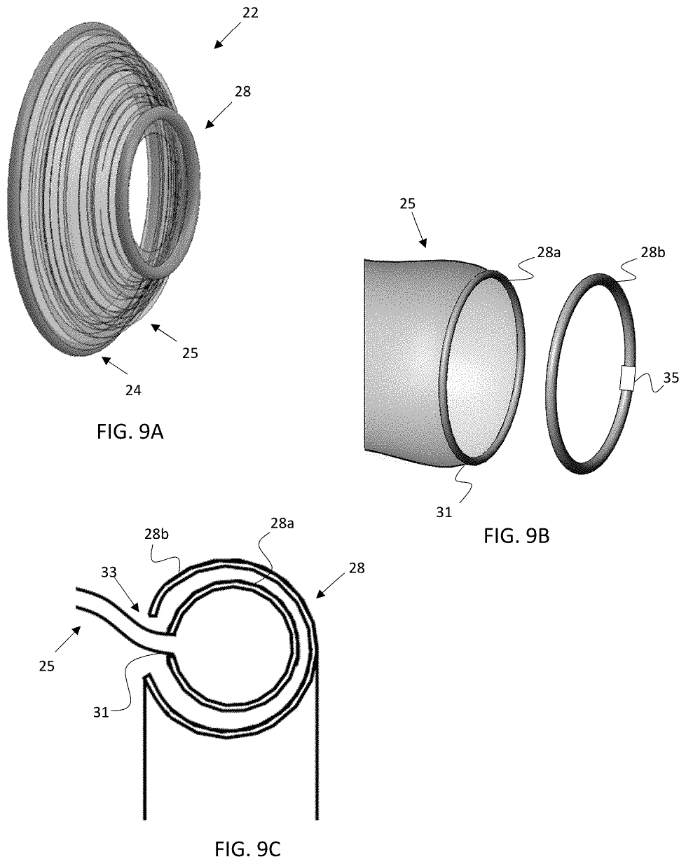

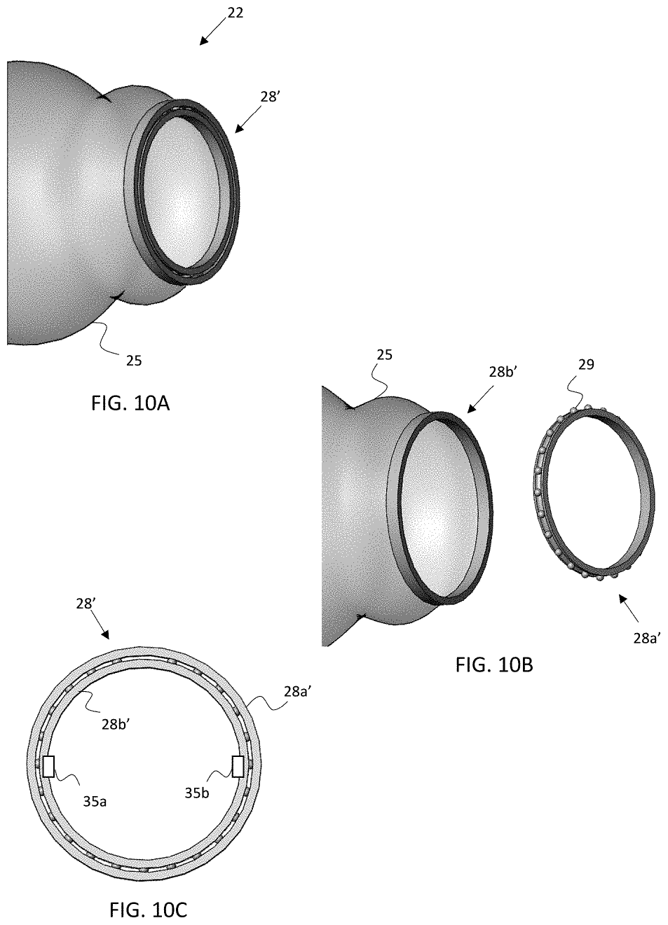

FIGS. 9A-10C, depict examples of a rotationally unconstrained ring 28 (i.e., “slip ring”). As previously described, a rotationally unconstrained ring 28 may be fixed to a portion (e.g., segment of the robotic arm 14, end-effector manipulator 18, or end-effector 20) of the robotic arm 14, and “slip” relative to any rotation of that portion of the robotic arm 14 to prevent the drape 22 or tube 25 from constricting (i.e., to prevent the drape 22 or tube 25 from rotating with the rotation of the robotic arm 14). For example, a part or a portion of a rotationally unconstrained ring 28 may be fixed to the end-effector manipulator 18 to axially constrain (i.e., prevent translational motion along an axis) the ring 28 to the end-effector manipulator 18. The rotationally unconstrained ring 28, now axially constrained to the end-effector manipulator 18, allows the drape 22 or tube 25 to ‘slip’ relative to any rotation of the end-effector manipulator 18 during a robotic task. In other words, the rotationally unconstrained ring 28 prevents the drape 22 or tube 25 from rotating with the rotation of the end-effector manipulator 18 while axial constraining the ring 28 to the end-effector manipulator 18. This allows the end-effector manipulator 18 to rotate an unlimited number of times without constricting the drape 22 or tube 25. It also allows the end-effector manipulator 18 to perpetually invert, by of a wrist joint of the robotic arm, without having the rotationally unconstrained ring 18 and drape 22 axially fall-off the robotic arm 14.

FIG. 9A depicts the drape 22 of FIG. 3 in an assembled state, FIG. 9B depicts the drape 22 in an exploded view, and FIG. 9C is a cross-sectional view of the rotationally unconstrained ring 28 and a portion of the tube 25. The rotationally unconstrained ring 28 includes an inner ring 28a and an outer ring 28b. The tube 25 of the drape 22 has a tube end 31 connected to the inner ring 28a. As best seen in FIG. 9C, the inner ring 28a is located inside the outer ring 28b and rotates relative to the outer ring 28b. The outer ring 28b may have a hollow interior to hold the inner ring 28a therein. The outer ring 28b may have an arced cross-section ranging between 181 degrees to 359 degrees to form a gap 33 between the arc ends. A portion of the tube 25 extends from the inner ring 28a and out through the gap 33 of the outer ring 28b. The outer ring 28b may further include one or more built-in connection mechanisms 35 (e.g., a joint, a clip, a clasp) to connect or interlock with a corresponding mechanism on a portion of the robotic arm 14 (e.g., end-effector 20, end-effector manipulator 18). To install the drape 22 on the robotic arm 14, the outer ring 28b is fixed to the end-effector manipulator 18 (or other distal portion of the robotic arm 14) to axially constrain the rotationally unconstrained ring 28 to the robotic arm 14. The outer ring 28b may be fixed to the end-effector manipulator 18 with the built-in connection mechanism 35 or with other connection mechanisms (e.g., adhesives, fastening elements (pins, screws, clamps, clasps, hook and loop)). The tube 25 is then draped over the robotic arm 14 to position the second ring 24 at a proximal portion of the robotic arm 14. During a robotic task, the fixed outer ring 28b rotates with the end-effector manipulator 18, and the inner ring 28a “slips” relative to rotation of the outer ring 28b to prevent the drape 22 or tube 25 from rotating with the end-effector manipulator 18. This allows the end-effector manipulator 18 to rotate an unlimited number of times without constricting the drape 22 or tube 25.

With reference to FIGS. 10A-10C, another example of a rotationally unconstrained ring 28′ (i.e., “slip ring”) is shown in the form of a bearing, where FIG. 10A is an assembled view thereof, FIG. 10B is an exploded view thereof, and FIG. 10C is a plan view of the rotationally unconstrained ring 28′. The rotationally unconstrained ring 28′ includes an inner ring 28a′ and an outer ring 28b′. The outer ring 28b′ is connected to a tube end 31 of the tube 25. The outer ring 28b′ and inner ring 28a′ form a bearing and may further include a race and a plurality of balls/rollers 29 to form a race bearing. The space between the inner ring 28a′ and the outer ring 28b′ may be sealed to form a sealed bearing to prevent contamination. The inner ring 28a′ may further include one or more built-in connection mechanisms (35a, 35b) to connect or interlock with a corresponding mechanism on a portion of the robotic arm 14 (e.g., end-effector 20, end-effector manipulator 18). To install the drape 22 on the robotic arm 14, the inner ring 28b′ is fixed to the end-effector manipulator 18 (or other distal portion of the robotic arm 14) to axially constrain the rotationally unconstrained ring 28′ to the robotic arm 14. The inner ring 28a′ may be fixed to the end-effector manipulator 18 with the built-in connection mechanisms (35a, 35b) or with other connection mechanisms (e.g., adhesives, fastening elements (pins, screws, clamps, clasps, hook and loop)). The tube 25 is then draped over the robotic arm 14 to position the second ring 24 at a proximal portion of the robotic arm 14. During a robotic task, the fixed inner ring 28a′ rotates with the end-effector manipulator 18, and the outer ring 28b′ “slips” relative to the rotation of the inner ring 28a′ to prevent the drape 22 or tube 25 from rotating with the end-effector manipulator 18. This allows the end-effector manipulator 18 to rotate an unlimited number of times without constricting the drape 22 or tube 25.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes may be made in the function and arrangement of elements without departing from the scope as set forth in the appended claims and the legal equivalents thereof.

1. A protective drape for a robotic arm having an end-effector manipulator and an end-effector, comprising:
a protective tube for encapsulating at least a portion of said robotic arm; and
a slip ring connected to a first end of said protective tube, wherein the slip ring is rotationally unconstrained to prevent the protective tube from rotating with a rotation of the end-effector or the end-effector manipulator when the slip ring is fixed to a portion of the end-effector or a portion of the end-effector manipulator.

2. The protective drape of claim 1 wherein the slip ring comprises:
a first member for fixating to the portion of the end-effector or portion of the end-effector manipulator to axially constrain the slip ring; and
a second member connected to the first end of the protective tube, wherein the first member rotates relative to the second member.

3. The protective drape of claim 1, wherein the first member is an inner ring and the second member is an outer ring.

4. The protective drape of claim 2, wherein the inner ring and the outer ring form a bearing.

5. The protective drape of claim 3 wherein the bearing is a sealed bearing.

6. The protective drape of claim 5, wherein the bearing is a ball bearing.

7. The protective drape of claim 1, wherein the first member is an outer ring and the second member is an inner ring.

8. The protective drape of claim 6, wherein the inner ring is located in a hollow interior of the outer ring.

9. The protective drape of claim 1 further comprising a second ring located at a second end of said protective tube, said second ring configured to fit around a proximal portion of said robotic arm.

10. The protective drape of claim 8 wherein said protective tube is foldable between said rotationally unconstrained ring and said second ring.

11. The protective drape of claim 8 wherein said rotationally unconstrained ring or said second ring forms a hermetic seal with said robotic arm.

12. The protective drape of claim 1 further comprising one or more draw cords located along a length of said protective tube.

13. The protective drape of claim 1 wherein said protective tube is made of paper, woven materials, fabrics, plastic films, plastic sheets, foils, or a combination thereof.

14. The protective drape 1 further comprising a blower for intraluminal pressure control.

15. The protective drape of claim 1 further comprising an access opening in a portion of said protective tube and a closure seal adapted to selectively seal said access opening.

16. The protective drape of claim 1 wherein the distal portion of the robotic arm comprises an end-effector or end-effector manipulator.

17. The protective drape of claim 1 wherein the first member further comprises one or more connection mechanisms to connect with a corresponding mechanism on the distal portion of the robotic arm.

18. The protective drape of claim 1 further comprising one or more sectional rings located along a length of the protective tube.

19. The protective drape of claim 1 further comprising an irrigation, vacuum, or air tube/line integrated with said protective tube.

20. A robotic system, comprising:
the protective drape of claim 1; and
a robotic arm having an end-effector and end-effector manipulator, wherein the slip ring is fixed to the portion of the end-effector or the portion of the end-effector manipulator.
